(19) United States
(12) Patent Application Publication
(10) Pub. No.: US 2006/0167432 A1
(43) Pub. Date: Jul. 27, 2006

(54) Reusable diaper and inside pad insert with optional shorts, pants, or skirt

(76) Inventor: Angela Susan Sigari, Marietta, GA (US)

Correspondence Address: Angela Sigari, 355 Briarwood Court, Marietta, GA, 30068, US

(21) Appl. No.: 11/287,629

(22) Filed: Nov. 28, 2005

Related U.S. Application Data
(60) Provisional application No. 60/631,496, filed on Nov. 30, 2004.

(51) Int. Cl. A61F 13/15

(52) U.S. Cl. 604/391

(57) ABSTRACT

A pants-type diaper (herein referred to as ‘diaperwear’) which combines an array of absorbent fabrics and polyurethane laminated material to create a new and unique diapering system while adding comfort, fashion, design, and convenience for both baby and caregiver. This diaperwear consists of a two or more layer waterproof diaper cover, absorbent disposable or non-disposable insert (pad, pre-fold, or other diaper), and optional outer clothing (shorts, pants, or skirt). Diaperwear is to be used as daytime wear. An adult version is also available for incontinence, and the pad insert alone can be placed inside undergarments and be sold separately for such.

### BACKGROUND OF THE INVENTION

The invention relates to washable, reusable diaperwear which consists of a two or more layer waterproof diaper cover, absorbent disposable or non-disposable insert (pad, pre-fold, or other diaper), and optional outer clothing (shorts, pants, or skirt). This invention came about as a result of testing to improve diapering. Several factors were taken into consideration including drying time after washing, leakage and wicking, comfort (especially around leg openings and waist), and aesthetics (so as the diaper is not only a diaper, it can actually be a piece of clothing with a diaper inside and functions with a dual purpose). Diaperwear is to be used as daytime wear for infants and toddlers, and pad insert (or other absorbent material) should be changed frequently. An adult version is also available for incontinence, and the pad insert alone can be placed inside undergarments and be sold separately for such.

### SUMMARY OF THE INVENTION

It is the object of this invention to provide an improved method of diapering along with an aesthetically fashionable diaper which can also serve as an actual piece of clothing in the form of shorts, pants, or shirt. This invention varies from any other diapering system on the market in that when waterproof diaper cover is used in conjunction with the layered washable, waterproof pad insert, this allows a dual waterproof barrier to help prevent leakage. Invention also varies from any other diapering system on the market in that it has the option of attached outer clothing.

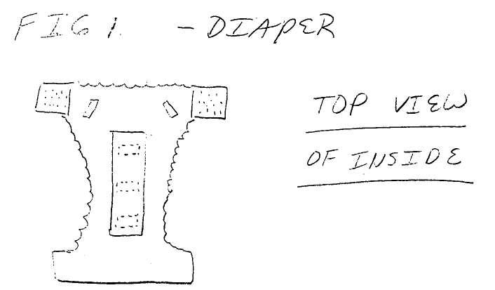

FIG. 1—DIAPER—top view of inside

All-in-one nondisposable diaperwear consists of a combination of diaper cover and inside diaper pad (used in lieu of a traditional diaper. Pad has option of being disposable or nondisposable), with option of attached shorts, pants, or skirt on the outside. Also optional, diaper cover may be converted into a multi-layer diaper by adding several layers of absorbent fabric in lieu of or in addition to, the diaper pad. For practical purposes herein, the above in any of these combinations will simply be referred to as “diaper”.

The diaper option without shorts, pants or skirt is an hourglass shaped two layer piece with extended winged tabs at the back waist; one layer of waterproof fabric on the inside and one layer of colorful solid or print fabric on the outside, with diaper pad inside next to baby's skin. When folded in half up over baby for diapering, wings at the top back waist of the diaper fold over the front waist and fasten to it with hook/loop; hook on the back inner waist winged tabs and loop on the front outer waist of diaper. An elastic strip is sewn around the entire perimeter of the diaper on the outside edge, holding the layers of fabric together. There are three horizontal strips of loop on the inside of the diaper to fasten to the three hook strips of the diaper pad. (Note: if the above is converted into a multi-layer diaper, layers of birdseye weave, terry and flannel are added on top of the waterproof fabric, next to baby's skin. Loop strips may or may not be added to this layering depending on customer's preference to use diaper pad in addition to this layering or not). There are two additional strips of loop near the winged tabs for foldover during laundering.

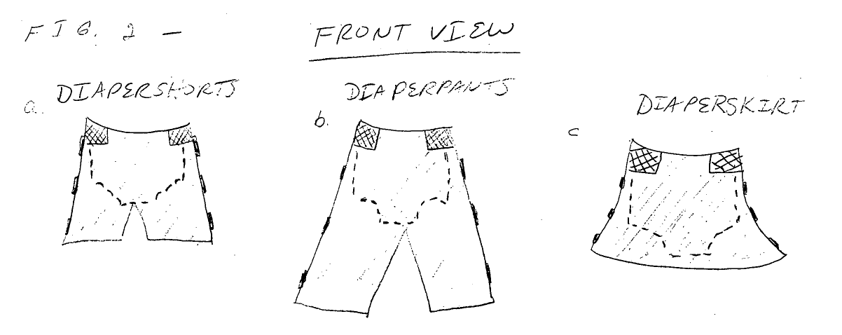

FIG. 2a—DIAPERSHORTS—front view

FIG. 2b—DIAPERPANTS—front view

FIG. 2c—DIAPERSKIRT—front view

The diaper with optional attached shorts, pants or skirt is the same as in FIG. 1 with additional pieces—the shorts, pants or skirt is either permanent or removable. The diaper is underneath the outer shorts, pants or skirt. The shorts, pants or skirt each consist of a front panel and back panel which are attached to the front and back of the diaper, with option of either permanently by being sewn on, or removable with hook/loop, buttons, snaps, etc. The skirt front panel is one rectangular piece. The skirt back panel is one rectangular piece. The shorts or pants front panel is composed of two pieces, sewn together in the middle from waist to crotch. The shorts or pants back panel is composed of two pieces, sewn together from waist to crotch. The shorts or pants front panel is attached to the shorts or pants back panel crosswise at the crotch, creating shorts or pants legs (openings). An elastic strip is sewn around the entire perimeter of the diaper, on the outside edge, with shorts, pants or skirt attached at the front and back waist, holding the layers of fabric together. The sides of the shorts, pants or skirt are fastened by means of hook/loop, buttons, snaps, frog closures, ties, etc. There are two additional strips of loop near the winged tabs for foldover during laundering.

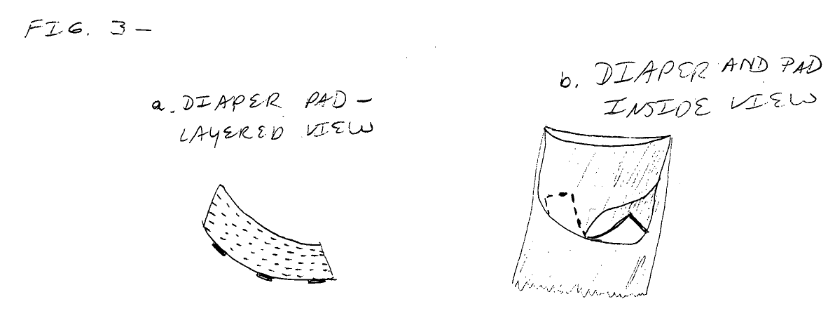

FIG. 3a—DIAPER PAD—layered view

FIG. 3b—DIAPER AND PAD—inside view

Diaper pad is either nondisposable or disposable, attached to inside of diaper by hook/loop. Nondisposable pad is composed of several layers of birdseye weave diaper cloth, terry cloth, flannel and water proof fabric. These layers are sewn together on four sides [rectangular], are available in two sizes, and have three horizontal hook strips attached on their waterproof underside so as to attach to the three strips of loop on inside of diaper. Disposable pad can be substituted instead, composed of disposable material with three disposable plastic horizontal strips of hook on underside of pad so as to attach to the three strips of loop on inside of diaper.

What is claimed is:

1) A reusable diaper(wear) comprising:
a) absorbent diaper pad insert
b) diaper cover
c) optional outer clothing

2) An absorbent diaper pad insert according to claim 1 wherein several layers of a combination of absorbent materials such as flannel, terry, birdseye weave, fleece, etc. are sewn together in a rectangular shape.

3) The absorbent article of claim 2 wherein one side of the pad is a waterproof, polyurethane laminated material.

4) The absorbent article of claim 2 wherein three strips of hook (hook/loop material) are sewn horizontally onto the waterproof side of the pad.

5) The absorbent article of claim 2 wherein pad can simply be pulled out when wet or soiled and replaced without having to wash entire garment.

6) The absorbent article of claim 2 wherein pad can be substituted with any other cloth diaper, generic (Chinese) prefold, or disposable pad which will fit into diaper cover to absorb fluid and catch waste.

7) A diaper cover according to claim 1 wherein two hour-glass shaped pieces of fabric, with extended winged tabs at the back waist, are sewn together, comprising:
a) outer layer in fashion print or solid
b) inner layer of waterproof, polyurethane laminated material.

8) The outer layer according to claim 7 has loop tabs on the front waist, so when folded in half up over baby, wings at the top back waist of the diaper fold over the front waist and fasten to it with hook/loop; hook on the back inner waist winged tabs and loop on the front outer waist of diaper.

9) The inner layer according to claim 7 has hook tabs on the back wings, so when folded in half up over baby, wings at the top back waist of the diaper fold over the front waist and fasten to it with hook/loop; hook on the back inner waist winged tabs and loop on the front outer waist of diaper.

10) The diaper cover according to claim 7 can be converted into a multi-layer all-in-one diaper by adding several layers of absorbent material (in lieu of or in addition to the diaper pad), on top of the waterproof layer.

11) The diaper cover according to claim 7 wherein an elastic strip is sewn around the entire perimeter of the diaper on the outside edge to hold the layers of fabric together and create elasticized areas around the leg openings, waist and back for comfort and to prevent leaks.

12) The diaper cover according to claim 7 wherein there are three horizontal loop strips (½″×2″ each) on the inside of the diaper cover to fasten to the three hook strips of the diaper pad insert.

13) The diaper cover according to claim 7 wherein there are two loop strips (½″×2″ each) on the inside of the diaper cover sewn in diagonally near the hook tabs on the back waist for “fold-over” of the hook tabs for laundering purposes.

14) The diaper(wear) according to claim 1 wherein optional shorts, pants, or skirt are attached to diaper cover.

15) The diaper(wear) according to claim 1 wherein optional shorts, pants, or skirt are attached at the front and back waist of the diaper cover either by being sewn on or by using options such as buttons, hook/loop, snaps, zipper, etc., thereby becoming permanent or removable.

16) The diaper(wear) according to claim 1 wherein skirt is composed of two separate panels; one in back and one in front.

17) The diaper(wear) according to claim 1 wherein shorts or pants are composed of four separate panels; two front pieces sewn together to form one panel in front and two back pieces sewn together to form one panel in back, then both pieces are sewn in middle at crotch to create leg openings.

18) The diaper(wear) according to claim 1 wherein the sides of the shorts, pants or skirt are fastened by means of hook/loop, buttons, snaps, etc.

19) The diaper(wear) according to claim 1 wherein a brief description of the several views of the drawings as required by 37 CFR 1.74 and 37 CFR 1.77 (b) (7) are below:
